(19) United States
(12) Patent Application Publication
(10) Pub. No.: US 2003/0104394 A1
(43) Pub. Date: Jun. 5, 2003

(54) Method and system for gene expression profiling analysis utilizing frequency domain transformation

(76) Inventors: Xudong Dai, Skokie, IL (US); Tong Fang, Old Bridge, NJ (US); Wei Xiong, Piscataway, NJ (US)

Correspondence Address: Robert A. Green, Esq., 17 Hampton Drive, Marlboro, NJ, 07746, US

(21) Appl. No.: 09/998,167

(22) Filed: Dec. 3, 2001

(51) Int. Cl. C12Q 1/68; G06F 19/00; G01N 33/48; G01N 33/50

(52) U.S. Cl. 435/6; 435/287.2; 702/20

(57) ABSTRACT

An iterative process to associate patterns embedded in the profiles of biological signals, including gene expression profile, protein profiles, with certain cellular status, functional stages and response to permutations. The biological signals, including gene expression profile, are converted into frequency domains using wavelet transform or other frequency transforms at different scales after rearranging the order of genes. These biological signals in the frequency domain are associated with certain cellular status, functional stages and response to permutation with neural network learning. An error rate is used to determine the optimal combination of wavelet function, scale and gene order. The information enriched gene group can be extracted from the frequency domain as well.

### BACKGROUND OF THE INVENTION

1. Field of the Invention

The present invention relates to the field of functional genomics. In particular, the invention relates to a method for extracting gene expression signature from gene expression profiling data based on frequency domain transformation using wavelets analysis.

2. Prior Art

The problem addressed by the present invention relates to the methodology to extract the gene expression signatures that are essential for functional genomic study, cancer diagnosis and drug response prediction. Identifying a gene expression signature that associates with any biological response or biological status of cell, tissue, organ and/or whole biological entity is an essential task for applications of gene expression profiling analysis. For example, using gene expression patterns in responding to hepatotoxcin, the liver toxicity of a potential drug can be predicted. To detect such gene expression patterns, various numbers of genes, from several hundreds to tens of thousands of genes, are measured to obtain a global gene expression profile, or interchangeably gene expression profile, by recently emerged technologies, such as cDNA microarray, high density oligonucleotide array (gene Chip), random optic fiber array and many other platforms. Then gene expression signatures embedded in the global gene expression profiles are extracted.

This invention is a method and a system for functional analysis and pattern recognition of gene expression profiles from microarray and other technical platforms. The core of this invention is a comprehensive approach using the wavelet and other transforms to transfer gene expression information into frequency domain and then extract the information associated with certain biological functions or biological status, which are essential for functional genomic study, cancer diagnosis and drug response prediction. A primary search, therefore, has been done using combination of key words such as “wavelet” or “Fourier” and “Gene” or “Expression”. No patent or patent application has been identified to have the combination of these words or concepts. The following six patents are related to the topic of gene expression profiling and data mining for the gene expression profiling.

U.S. Pat. Nos. 5,707,807, 6,136,537, 6,174,683, 6,229,911 are different from the present invention. These U.S. patents are methods for obtaining the gene expression profile raw data, the present invention, however, is a method or system for analyzing the gene expression profiling data and provides a method for extracting biological information from the raw data.

U.S. Pat. No. 6,245,517 discloses a method to collect the intensities of the microarray data and transfer the intensities into relative expression levels by calculating the ratio of the expression for each individual gene under two different conditions. It differs from the present invention that transfers the gene expression profile consisting of relative or absolute levels of all the genes into frequency domain and associates such signals in the frequency domain with the biological functions.

U.S. Pat. No. 6,185,561 covers a method to obtain gene expression profile data and establish a database with such data providing the raw gene expression profile data. The present invention analyzes the raw gene expression profile data to provide a method for extracting biological information from it.

In general, a thorough search of the patent documents and patent application documents does not uncover any previous patent that overlaps the present invention. The following papers, however, were found in publications [1-7] related to the subject matter of the current invention. The comparisons between these papers and the present invention are described as follows:

Four [4-7] papers reported a variety of applications of wavelet or other transforms on individual string of DNA sequence which consists of a string of combination of A, C, G, and T nucleotides as basic components. Our invention differs from these papers based on the following reasons:

1) The work described in these four papers [4-7] involves the study of individual DNA sequences. They are not related to the analysis of gene expression profiles. The present invention differs from the four reported works in that the present invention studies gene expression data, which consists of hundreds of thousands of genes, and retrieves biological functional information from the data while the work revealed in the four papers only study the structural characteristics of DNA sting, which could be part of an individual gene, or a sequential combination of a gene and prior or post gene components.

2) The DNA string has a sequential nature and such inherent spatial information is directly transformed into frequency domains without any permutation. The global gene expression profiles do not have any intrinsic spatial information naturally. One of the novelties of the proposed invention is to rearrange the order of all genes in the gene expression profile so that the biological function and status can be associated with the transformed information in the frequency domain.

Three papers [1][2][3], do show the utility of the transformed frequency domain information from gene expression data The proposed invention, however, is novel in such a way that:

1) The present invention can transform gene expression profile from any experiments and associate the transformed profiles with the biological functions or cellular status. Specifically, the present invention uses each gene as an element of the individual global gene expression profile that reflects the biological function or status of a cell or a tissue. Instead of analyzing an individual gene's expression at multiple time points or physical locations, we study a snapshot consisting of thousands of genes at an individual moment and associate such a picture with the cellular function or status. There is no inherent order among all these genes but the rearrangement of the order of individual genes is allowed and is very useful for extracting patterns associated with certain biological functions or cellular status in the current invention.

The methods reported by Myasnikova, et al and Klevecz and Dowse can only be applied to two special cases in gene expression analysis, namely to the time serial study and to the spatial coordinately expressed genes. To analyze genes using their methods, it is required for genes to have certain temporal or spatial relationships. For example, Klevecz and Dowse's approach relies on the time course of the cell cycle. The wavelet is used to transfer the time course of individual gene into the frequency domain. Then genes are separated into different groups according to their temporal course revealed by the transform. Genes whose expressions do not share the same expression time course cannot be grouped into biologically meaningful clusters by Dr. Klevecz's method. Similarly, the study by Myasnikova, et al [1] about the expression patterns of Drosophila (fruit fly) segmenting genes with wavelet transforms depends on the spatial information associated with the segmenting genes. These genes are naturally expressed in a sequential manner from the head of a fruit fly to the tail of the fruit fly. With the wavelet transform, the spatial expression level of individual gene is transformed and then the genes that express at the same locations of the fruit fly are divided into the same group according to the transformed signal in frequency domain. Genes whose expressions are not coordinated with the segmentation of the fruit fly cannot be clustered into meaningful groups by Dr. Myasnikova's method.

The expression profiles transformed by both works are an individual gene's expression level at either different time points or physical locations as elements. Their work depends on the time course or spatial information to associate each individual gene according to the temporal and spatial information. The order of the time points and physical locations cannot be changed. Gene expression profiles that are not related to time course or do not have spatial information, such as predicting drug toxicity from an individual gene expression profile, cannot be studied by any of their approaches, but they can be studied by the proposed invention. The current invention, therefore, demonstrates significant difference from those works described above and can not be derived from any extension or any simple combination of reported work described above.

2) The third study by Jornsten and Yu [2] reports a method to reduce the size of microarray image information with wavelet and other transforms. It transfers the two-dimensional image data into a frequency domain and compresses the data by reducing the redundant information without losing information. The differences between the present invention and the third study are:

a) The proposed invention analyzes the gene expression profile and uses the transformed data to extract functional information from the gene expression profile, but not the raw image of the microarray;

b) The gene expression profiles analyzed in the proposed invention are a collection of quantified data from a group of microarray images, but not from an individual image as the reported approach does.

c) In the present invention the information about biological function or status can be associated with the expression profiles. But in the reported study only the image information can be retained from individual microarray with smaller file size.

d) In the present invention, genes are rearranged using a specific method so that the transformed signal of gene expression profile can be associated with certain biological functions. While in the reported study the raw image data is directly transformed into frequency domain and is not associated with any functional information.

### References

U.S. Patent Documents

1) U.S. Pat. No. 6,229,911, Balaban, et al. May 8, 2001 “Method and apparatus for providing a bioinformatics database’

2) U.S. Pat. No. 6,185,561, Balaban, et al. Feb. 6, 2001 “Method and apparatus for providing and expression data mining database’

3) U.S. Pat. No. 6,136,537 Macevicz Oct. 24, 2000 “Gene expression analysis”

4) U.S. Pat. No. 6,174,683 Hahn, et al. Jan. 16, 2001 “Method of making biochips and the biochips resulting therefrom’

5) U.S. Pat. No. 5,707,807, Kato Jan. 13, 1998 “Molecular indexing for expressed gene analysis”

6) U.S. Pat. No. 6,245,517, Chen, et al. Jun. 12, 2001 “Ratio-based decisions and the quantitative analysis of cDNA micro-array images’

### Publications in Public Domain

[1] Myasnikova E, Samsonova A, Kozlov K, Samsonova M, Reinitz J. “Registration of the expression patterns of Drosophila segmentation genes by two independent methods.” Bioinformatics. 2001 January; 17(1):3-12.

[2] Jornsten R. and Yu B. “‘Comprestimation’ Microarray Images in Abundance” Conference on Information Sciences and System. Princeton University. Mar. 15-17, 2000

[3] Klevecz R R, Dowse H B. “Tuning in the transcriptome: basins of attraction in the yeast cell cycle”. Cell Prolif. 2000 August; 33(4): 209-18.

[4] Dodin G, Vandergheynst P, Levoir P, Cordier C, Marcourt L. “Fourier and wavelet transform analysis, a tool for visualizing regular patterns in DNA sequences” JTheorBiol. Oct. 7, 2000 ; 206(3):323-6.

[5] Morozov P, Sitnikova T, Churchill G, Ayala F J, Rzhetsky A. “A new method for characterizing replacement rate variation in molecular sequences. Application of the Fourier and wavelet models to Drosophila and mammalian proteins.” Genetics. 2000 January; 154(1):381-95.

[6] Altaiski M, Mornev O, Polozov R. “Wavelet analysis of DNA sequences.” Genet Anal 1996 March; 12(5-6): 165-8

[7] Lio P, Vannucci M. “Finding pathogenicity islands and gene transfer events in genome data. Bioinformatics. 2000 October; 16(10):932-40.

The prior art of analysis methods being used to classify gene expression profiles includes clustering, two-way clustering, support vector machine and principal component analysis (PCA). Except for the PCA method, all other methods use part of or all genes from global gene expression profiles as feature vectors for prediction. Due to the limitation of high dimensional gene feature vectors, smaller number of observations and the requirement for identifying feature genes, the accuracy of predictions using these methods may vary from one case to another.

To extract gene signatures that can separate gene expression profiles from predetermined different classes of permutations or biological status from a set of relatively small size of observations, a method for gene feature extraction is needed to determine the most distinctive feature vectors that can generate a group of feature genes from all genes used for global gene expression profiling. However, no such method has been revealed by any prior literature or patents for gene expression profiling analysis.

The present invention provides a method and a computer system to utilize frequency domain feature extraction and reduction for gene expression signature detection and permutation classification. It is shown that in the detailed description of preferred embodiments the frequency transform of global gene expression profile can maintain the amount of separability while reducing the number of feature vectors and necessary observations.

The first step in our proposed method is to iteratively select an order of genes that has the characteristics desired for the particular application from a set of ordering methods, including random order. Second, an optimal order of genes is determined according to the preset criteria of class prediction error rate measured by comparing the difference between known classes of given permutations and the predicted classes obtained by classification of these permutations. Then, from Wavelet transform and other frequency transforms including Discrete Fourier Transform (DFT), but not excluding other transforms, a transform method is iteratively selected to be used to extract gene feature vectors. An optimal transform method can be determined according to the preset criteria of class prediction error rate measured by comparing the difference between known classes of given permutations and the predicted classes obtained by classification of these permutations. After the optimal order of genes and optimal frequency transform method are determined, the ordered group of genes that is essential for the classification of global gene expression profiles can be transformed by the selected frequency transform method. The gene signature can thereafter associates with biological function, cellular status, disease or disorders in the frequency domain.

The above description is exemplary only and should not be deemed limiting our invention in any way for applications in functional studies of genes and their profiles.

### SUMMARY OF THE INVENTION

It is an object of the present invention to provide a means for associating gene expression profile with certain phenotypes and cellular status, including, but not excluding, cell proliferation, death, differentiation and any biological response after certain permutation.

It is another object of the present invention to provide a means for extracting gene expression signature or pattern from frequency domain to permit reliable clustering of genes, permutations or biological status and to provide reproducible classification for permutations, biological status and functional annotation of genes.

It is also another object of the present invention to provide a means for determining optimal orders of genes to permit reliable and reproducible clusters and classification of genes, permutations or biological status represented by gene expression profiles.

It is yet another object of the present invention to provide a means for determining features of transformed signal in frequency domain from gene expression profiles to permit maximal separation among gene profiles representing different functions, pathways, biological status and permutations.

It is finally another object of the present invention to provide a means for extracting as few transformed features in frequency domain as needed to permit reliable and reproducible clusters and classification of genes, permutations or biological status represented by gene expression profiles.

These and any other objects of the present invention are materialized by a computer system and method for performing gene list reordering and optimization, frequency domain transformation with wavelet and/or DFT, feature extraction and recovery for clustering and classification of gene expression profile.

The present invention comprises an iterative process to optimize the order of gene list and to determine the appropriate wavelet function and combination of scales of this wavelet function so that the predicted classification of gene profiles can represents full spectrum of different classes of permutation and biological status. The error rate of classification is measured using a training data set and an evaluation data set. The optimal order of genes and the optimal setting of wavelets are determined for class prediction when the feature vectors in frequency domain transformed from such gene order with selected wavelet setting can provide the lowest classification error or lower than pre-set error rate criteria.

The present invention needs to generate a library of gene lists from pre-selected orders, hereinafter, referred to as “gene ordered lists”. Then a gene expression profile from observation or permutation is rearranged according to the selected gene ordered list and hereinafter referred to as an “ordered gene expression profile”. The ordered gene expression profile is composed of the relative gene expression levels.

The present invention yet needs to generate a library of pre-selected wavelet functions, hereinafter, referred to as “wavelets”. Wavelets are chosen for inclusion in the library according the preestablished criteria for specific application.

The parameters of coefficients of the wavelets are selected to produce the transformed input data from ordered gene expression profiles to provide classification accuracy and computational efficiency required by applications. Error rate of the resultant wavelet implemented digital filtration is examined for each of the scales of transforms. The error rate of classification is calculated for each set of wavelet transform. The error rate of each individual set of wavelet transform is recorded for determination of the optimal set of wavelet parameters and gene orders thereafter.

When all gene order and wavelets have been applied, the error rate of classification is examined with a set of training dataset and evaluation dataset. The estimated classification is predicted by backward propagation/MLP, the error rate of the prediction is calculated as the percentage of incorrect prediction classes from total prediction classes using bootstrap and/or crossover evaluation methods.

An appreciation of the objects of the present invention and a full understanding of its structure and method of operation may be established by studying the following description of the preferred embodiment and by referring to the accompanying drawings.

### BRIEF DESCRIPTION OF THE DRAWINGS

FIG. 1 is a functional flow chart of the present invention.

FIG. 2 is an illustration of an ordered gene expression profile in original order.

FIG. 3 is an illustration of a reordered gene expression profile in rearranged rank order.

FIG. 4 is a Back propagation/MLP validating plot for error rate estimation in the training process.

FIG. 5 compares the classification error rate by RMS among random order genes and rank order genes with 64 and 256 feature variables.

### DESCRIPTION OF THE PREFERRED EMBODIMENTS

The present invention is a method and a computer system for implementing the method to extract gene expression signature from gene expression profiling data based on frequency domain transforms using wavelet analysis.

As it is known, the term “wavelet transform ” is based on wavelets as well as to a second subset of operations referred to as “wavelet packet transform” based on wavelet packets. Unless otherwise suggested by context the term “wavelet transform” as used in this specification and the claims refers to both subsets, that is, to both wavelet transform and wavelet packet transform. Similarly, “wavelets” will refer to both wavelets in the narrower sense of the term as well as wavelet packets.

The present invention extracts the features so that the error rate of classification of gene expression profiles, which represent different classes of permutation and biological status, is minimize or meet the preset criteria.

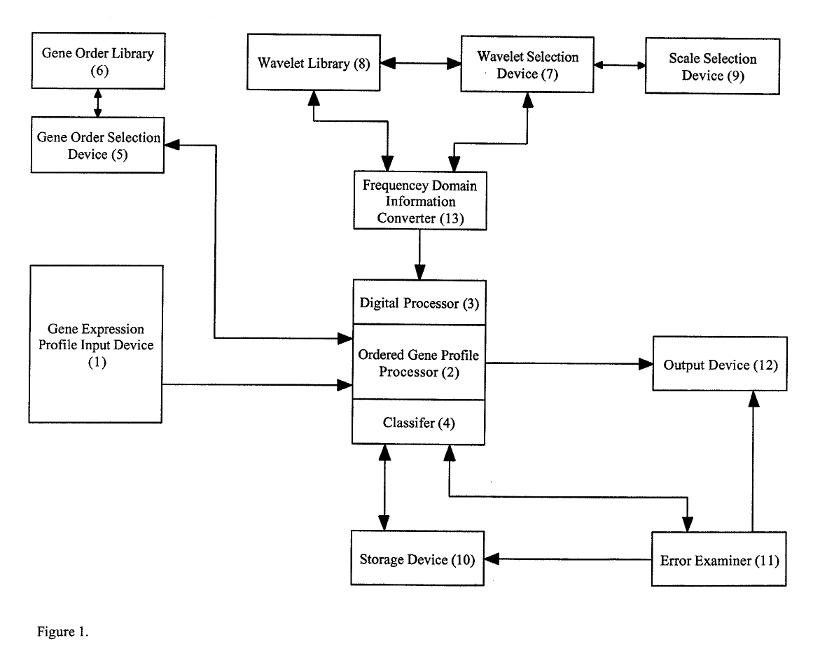

The apparatus and the methodology of the present invention are depicted in FIG. 1. The present invention comprises a gene expression profile input device (1), a gene order selection device (5), a gene order library (6), wavelet selection device (7), a wavelet library (8), a scale selection device (9), an ordered gene profile process device (2), digital processor (3), a classifier (4), an output device (12), an error examination device (11) and a storage device (10).

The global gene expression profiles are captured and stored in the gene expression profile input device (1), which may be internal or external to the digital processor (3) or a combination of both internal and external. The raw data in the gene expression profile input device is converted into a relative or normalized gene expression profile. The gene order library (6) includes, but does not exclude original order, rank order, Euclidean distance order and correlation order. The gene order selection device (5) may be, for instance, a keyboard or a computer program subroutine connected to with and between itself the gene order library (6) and the ordered gene profile process device (2) to generate ordered relative gene expression profiles.

The ordered relative gene expression profiles are then transformed by wavelet analysis. To perform wavelet transform, a set of wavelet mother functions have to be selected from the wavelet library (8) by a wavelet selection device (7), which may be, for example, a keyboard, or a computer program function or subroutine. In addition, an appropriate scale for each selected wavelet function is determined by the scale selection device (9), such as a keyboard or a computer program subroutine.

After wavelet transform, the ordered relative gene expression profiles from a set of training datasets are used to train an MLP classifier (4) in the digital processor (3) with cross testing or bootstrap testing. Training stops when preset error rate criteria are met. Utilizing cross testing or bootstrap, the accuracy of the trained MLP classifier is determined by an independent set of evaluating data set after wavelet transform (11). The error rates for each set of wavelet setting and MLP setting are stored in storage device (10), including memory and hard drive. Also, they are output to the output device (12). The output device may be the computer screen or the printer. When the error rate with a set of wavelet setting reaches the preset criteria or the lowest level for the selected order, the selection of wavelet and its scale stops for that gene order selection. The trained classifier or the resulting clusters are subjects to validation with another set of independent dataset with the previously selected order and wavelet transform.

The pseudo code of extracting the gene expression signature from gene expression profiling data based on frequency domain transform using wavelets is shown below

Input gene expression profile

Convert gene expression profile into relative or normalized gene expression profile

Select a gene order

Convert relative gene expression profile into ordered relative gene expression profile

Do while (the iteration number<preset maximum iteration OR the error rate of classification>preset error rate)

Select a wavelet function

Do while (the iteration number for selecting scale<=preset maximum iteration for selecting scale)

Select a scale

Transform the ordered relative gene expression profile with the selected wavelet and scale;

Train a MLP classifier with the wavelet transformed gene expression profile from a set of training data set using cross validation and/or bootstrap validation;

Evaluate the trained MLP classifier with the wavelet transformed gene expression profile from a set of evaluating date set using cross validation and/or bootstrap validation;

Calculate the estimated error by averaging the individual classification errors for whole evaluating data set;

If (estimated error<preset error) then

Exit;

Else

Continue

Determine the scale that generates the lowest estimated error for the selected wavelet function

Determine the wavelet function and scale that generate the lowest estimated error for the selected wavelet function.

Validate the classifier or cluster with another set of independent data

Referring to FIG. 1 and the pseudo code, a library of wavelets is required. The wavelet functions for a particular type of application data and classifier can be obtained from sources such as Daubechies, as discussed with details in following sections:

Wavelet transform provides a precise and unifying framework for the analysis and characterization of signals or data in spatial/frequency domain. Therefore, wavelet analysis can process data at different scale, resolution, or frequency.

Wavelet selection device (7): The device may be a keyboard and/or a computer program subroutine or function that allows different forms of wavelet functions, including but not excluding, discrete wavelet transform or discrete wavelet packet transform, selected from the wavelet library device (8). In the discrete wavelet transform (DWT), the original data is successively decomposed into components of lower resolution (frequency), while the high frequency components are not analyzed any further. However, in the discrete wavelet packet transform (DWPT), the original data is successively decomposed into components of both low resolution (frequency) and high resolution (frequency) components.

Wavelet library device (8): There are different types of wavelet families whose qualities vary according to several criteria, such as time and frequency localization, symmetry, vanishing moments, orthogonality, regularity, exact reconstruction and discrete analysis ability. In our method and system for gene expression profiling analysis, the criteria of time and frequency localization, orthogonality, exact reconstruction and discrete analysis ability are more important. Therefore, only the Daubechies wavelet family, the Symlet wavelet family, the Coiflet wavelet family and the Meyer wavelet family are components of our wavelet library. We use the Daubechies wavelet function to be our default wavelet function due to its good properties in time and frequency localization, orthogonality and computational efficiency.

Scale selection device (9): The wavelet decomposition level directly corresponds to the resolution in frequency domain. For scale level N, the signal or data can be decomposed to (N+1) frequency sub-bands by using 1-dimensional discrete wavelet transform or 2N frequency sub-bands by using 1-dimensional discrete packet transform. Therefore, high wavelet decomposition level corresponds to high resolution and high feature vector dimension. In the example of preferred embodiments of the current invention, we select the scale level from 6 to 8.

Frequency domain information converter (13): In the example of the preferred embodiment of the proposed invention, the energy features are used to represent the gene expression pattern information in frequency domain. The energy measure X_{J} of a frequency subband j is defined as:

$${X}_{j} = \frac{1}{{m}_{j}} {{\sum }^{{m}_{j}}}_{i = 1} {y}_{ij}^{2}$$

where yᵢⱼ is i^{th} wavelet coefficient of subband j and mⱼ is total number of wavelet coefficients of subband j. In the normalized energy measure, each wavelet coefficient is centered on the mean. That is

$${x}_{j} = \frac{1}{{m}_{j}} {{\sum }^{{m}_{j}}}_{i = 1} {( {y}_{ij} - {μ}_{j} )}^{2}$$

Where

$${μ}_{j} = \frac{1}{{m}_{j}} {{\sum }^{{m}_{j}}}_{i = 1} {y}_{ij}$$

By way of the above example and as demonstrated in one preferred embodiment of the present invention, wavelets were selected from (8).

For one of the preferred embodiments of the present invention, a data set with 70 observations belonging to two classes is divided into a training data set with 38 observations and an evaluating date set with the other 32 observations in order to train and evaluate the classifier. To reduce the high dimension of the each gene expression profile, consisting of 7129 genes, a scale of 6 or 8 was selected to retain enough information.

For one of the preferred embodiments of the present invention, two hidden layers of neural network were applied. The first hidden layer includes 3 neurons and the second hidden layer includes 10 neurons. The preset minimal estimated error is selected to be zero.

For one of the preferred embodiments of the present invention, the gene order is selected as the original order. To initiate the process by the preferred embodiment of the present invention, the gene expression profiles are obtained from a gene expression profile input device (1) which may be a microarray or gene scanner or a storage device containing data received from a microarray or gene chip scanner or a sensor that directly or indirectly detects the level of gene expression in objective samples.

The present invention requires that the user initiate gene order selection, wavelet selection and scale selection via a gene order selection device, wavelet selection device and a scale selection device. The selection device (5), (7) and (9) may be a keyboard and/or a computer subroutine triggered automatically or manually by the user.

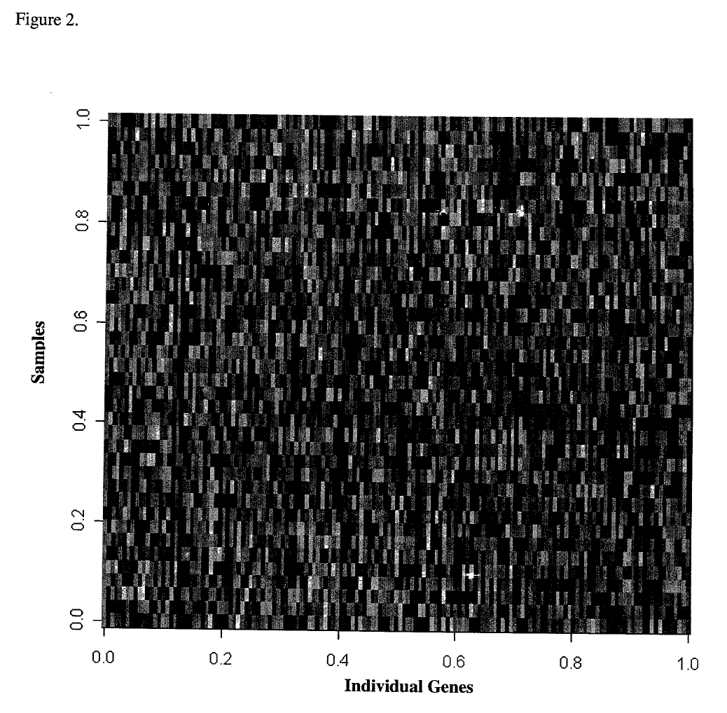

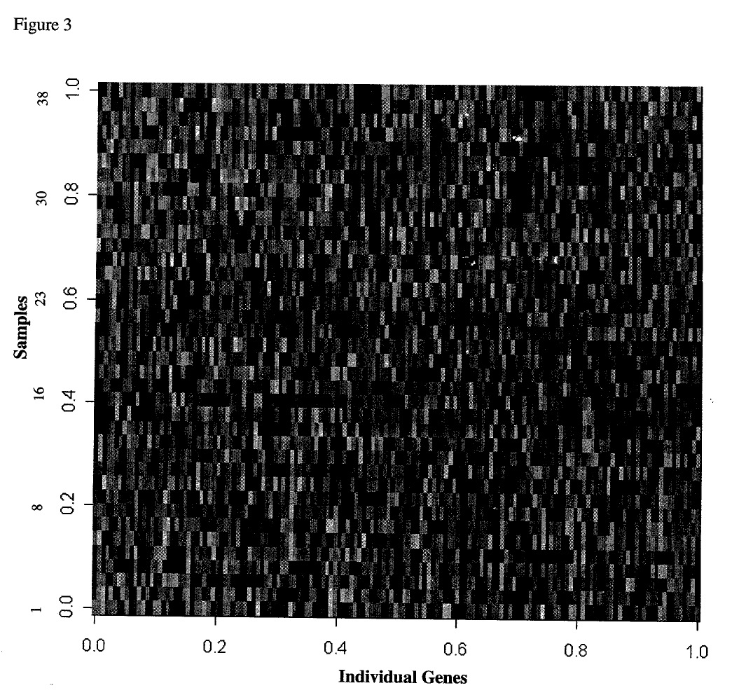

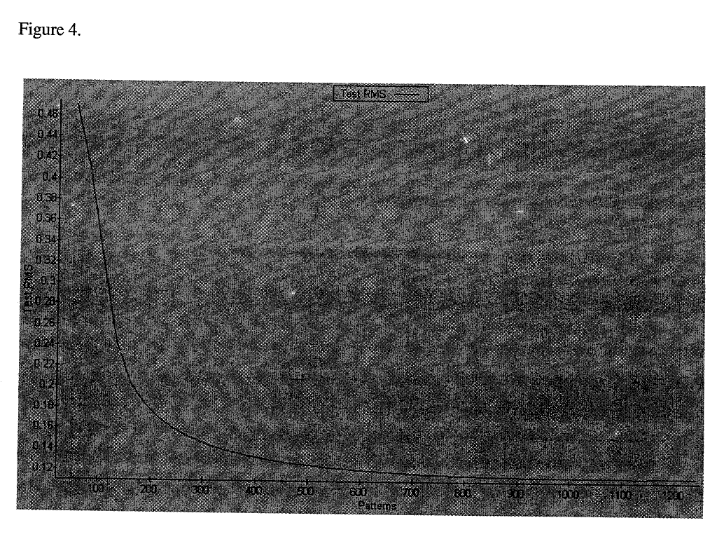

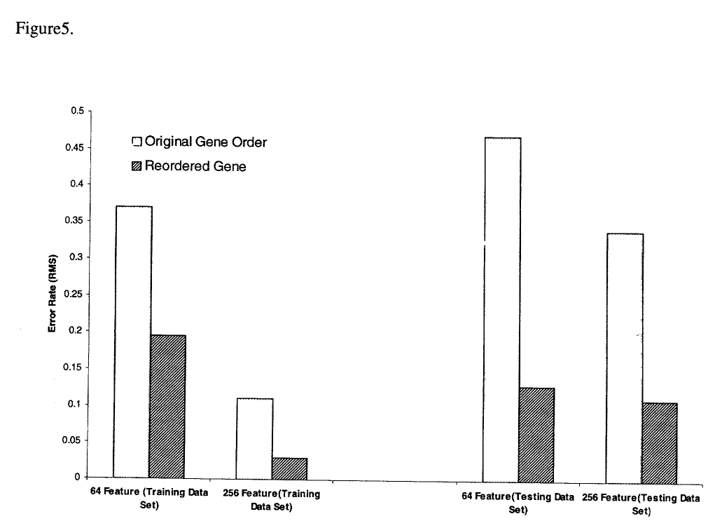

Using the present invention method implemented and automated by the system of the invention, the ordered gene expression profiles with original order and order in accordance with rank order are illustrated in FIG. 2 and FIG. 3. The two-dimensional pseudo color scaled the maps in the FIGS. 2 and 3 represent the global gene expression profiles in the original order (FIG. 2) and the rearranged order (FIG. 3). In both figures, the x-axis presents hundreds of genes being monitored and the y-axis presents the different specific experiments (samples). The gradient of the color indicates the value of an expression level for a certain gene of a certain experiment. After rearranging the order of genes in FIG. 2, a pattern of the expression profile emerges in FIG. 3. The results of using wavelet transform for feature extraction on a cancer diagnosis classification application with both original gene order and rank ordered gene order are illustrated in FIGS. 4 and 5. Specifically, FIG. 4 shows the estimated error of a testing data set from rank ordered gene list using Backpropagation/MLP in training process. The x-axis represents the number of trainings that has been done for the classifier. The y-axis indicates the estimated error, RMS, from the testing data set. As illustrated by the rapidly dropped error curve, a reproducible classifier with an estimated error rate lower than 0.12 can be obtained within 500 times of searching and training from the re-arranged data. The difference between the original order expressing profile data and rearranged profile data becomes more clear when the estimated errors from different level wavelet transform are compared (FIG. 5). The Error rates for the classification from both training data set (left panel) and testing data set (right panel) are compared between original profile and the rearranged profile with 64 and 256 wavelet feature vectors. Under both conditions, the error rate dramatically decreased from 37.5% to 20.0% for 64 features, 12.5% to 2.5% for 256 features in training data set and 47.5% to 17.5% for 64 features and 37.5% to 10.0% for 256 features respectively. It shows how the estimated error depends on the number of scale in the same classification of tissue samples represented by their gene expression profiles. Moreover, the gene expression profiles from rank ordered gene lists lead to lower error rate. The figure illustrates the feasibility and advantages of the present invention for association of certain cellular status or reaction with gene expression profiles.

Compared with the other approaches for classification and cluster of gene expression profile, the present invention has following advantages:

1) Low estimated error for high dimensional gene features and few observations from different classes of permutation and biological status.

2) Adjustable scale and computational complexity for classification and clustering applications on gene expression profile analysis

3) High adaptability with her feature reduction methods for MLP or other neural network algorithms on wavelet transformed signal. For example, the sensitivity analysis of individual coefficients for classification can be directly applied to the trained classifier and determine the most significant coefficients. The corresponding genes can be recovered by inverse wavelet transformation.

4) Insensitivity to classifier algorithms. Classifiers sensitive to high dimensional feature vectors may be used for gene expression profile analysis after the wavelet transformation in low scale provided enough data points in the training dataset.

The methods, classifiers, structures disclosed herein demonstrate the principles of the present invention. The invention may be embodied in other specific and derived forms without departing from its principal or essential characteristics. The elicited embodiments herein are to be treated in all respects as exemplary and illustrative rather than explicit and restrictive. Therefore the appended claims rather than the foregoing description define the range of the invention. Any modification and alteration to the embodiments described herein that are consistent with the meaning and belong to the scope of equivalence of the claims are included within the range of the invention.

### FIGURE LEGEND

FIG. 1. The flow chart of the invention

FIG. 2. Illustration of gene profile in original order (random order).

FIG. 3. Illustration of gene profile according to rank order (reordered genes).

FIG. 4. Decreasing of error rate for the test dataset during the progress of training.

FIG. 5. Comparison of the error rate between the data set in random gene order or rank gene order with 64 or 256 feature variables.

We claim:

1) The method of detecting gene expression pattern comprises selecting a number of genes from gene expression profiles.

2) The method defined in claim 1 and further extracting gene expression signature embedded in gene expression profiles.

3) The method defined in claim 1 and extracting the gene expression signature from the gene expression profile comprises several hundreds to tens of thousands genes which are measured by means of cDNA microarray, high density oligonucleotide array, random optic fiber array, and other platforms

4) The method of extracting gene expression signature, according to claim 2, wherein the gene expression signature from the gene expression profile is associated with biological functions or biological status.

5) The method defined in claim 1 including the step of performing frequency domain transforms using transforming functions comprising wavelets.

6) An apparatus wherein gene expression profile is processed to provide a plurality of gene expression pattern to permit maximal separation among gene expression pattern, said gene expression signature representing different biological functions.

7) The apparatus defined in claim 6 comprising devices for extracting gene expression signature from frequency domain to permit clustering of gene associated with permutations of biological function and status; and to provide reproducible classification for permutations, biological status and functional association of genes.

8) Apparatus defined in claim 6 including apparatus for processing gene expression profiles comprising a gene order library coupled to a gene order selection device;
a gene expression profile input device;
an ordered gene profile processor;
said gene order selection device and said gene expression profile input device both coupled to said gene expression profile processor;
an output device;
said ordered gene profile processor being coupled to said output device;
and a wavelet library and wavelet selection device being coupled through a frequency domain converter to said ordered profile processor.

9) The apparatus defined in claim 8 including an output device and an error examiner coupled to said output device, said error examiner being coupled through a storage device to said ordered profile processor.

10) A method of extracting gene expression signature based on a frequency domain transformation using wavelets comprises:
forming an input gene expression profile as training and testing profiles;
converting the gene expression profile into relative gene expression profile; selecting a gene order;
converting the relative gene expression profile into an ordered relative gene expression profile;
selecting a wavelet function;
selecting a scale;
transforming the ordered relative gene expression profile with the selected wavelet and scale;
training a classifier by classification method, comprising MPL or Bayesian neural network, with the wavelet transformed gene expression profile;
forming an ordered relative gene expression profile with a set of validating data with the same scale and wavelet used to form the training and testing profiles;
calculating an estimated error with the trained classifier.

11) The method defined in claim 10 and further determining optimal orders of genes to permit formation of reproducible clusters and classification of genes and permutations of biological states represented by gene expression profiles.

12) The method according to the claim 10 wherein an iterative procedure is utilized with different gene orders, wavelet functions and scales to determine the order of genes, the wavelet function and scale for the obtained classifier that demonstrates the lowest estimate error for classification of validating data set.

13) The method defined in claim 12 and converting the ordered relative gene expression profile into frequency domain at different scale after rearranging the order of genes.

14) The method according to claim 12 wherein a scale is selected for frequency domain transform of the gene expression profiles to permit maximal separation among gene expression signature representing different functions.

15) The method defined in claim 12 wherein said wavelet function is selected from the selecting device including a keyboard and a computer program subroutine function.
